(19) United States
(12) Patent Application Publication
(10) Pub. No.: US 2026/0033721 A1
(43) Pub. Date: Feb. 5, 2026

(54) Created Cavity Biometric Sensor

(71) Applicant: Incora Health, Inc., Greenville, SC (US)

(72) Inventors: Lindsey Calcutt, Greenville, SC (US); Matthew Gevaert, Greenville, SC (US); Theresa Gevaert, Greenville, SC (US); Tony Kobet, Clemmons, NC (US); Brad Long, Mocksville, NC (US); Dennis Whitley, Cooleemee, NC (US)

(21) Appl. No.: 18/995,393

(22) PCT Filed: Jul. 15, 2023
(86) PCT No.: PCT/US2023/027859

§ 371 Date: Jan. 16, 2025

Related U.S. Application Data
(60) Provisional application No. 63/389,891, filed on Jul. 17, 2022.

(51) Int. Cl. A61B 5/00; A61B 5/01; A61B 5/0205; A61B 5/021; A61B 5/11; A61B 5/145; A61B 5/1455

(52) U.S. Cl. CPC A61B 5/0028; A61B 5/0008; A61B 5/01; A61B 5/02055; A61B 5/021; A61B 5/1118; A61B 5/14503; A61B 5/14532; A61B 5/14551; A61B 5/6816; A61B 5/6838; A61B 2560/0214; A61B 2562/06

(57) ABSTRACT

Disclosed are devices, systems and related methods for wearable devices that sense various anatomical conditions and/or metrics of a wearer, including measuring and optionally transmitting this data to a computing device which analyzes the data to determine various physiological conditions within and/or in proximity to a wearer's body. The device comprises a central body having a first sensor and a body piercing post with a second sensor, wherein the sensors measure a first and second physiological condition respectively of the wearer continuously or periodically using specified time intervals.

### CROSS-REFERENCE TO RELATED APPLICATIONS

This application claims priority to and benefit thereof from U.S. Provisional Patent Application No. 63/389,891 filed Jul. 17, 2022, titled “CREATED CAVITY BIOMETRIC SENSOR,” the disclosure of which is incorporated by reference herein in its entirety.

### TECHNICAL FIELD

The present invention relates to wearable devices and associated system components that sense, measure, transmit, and present various data, including data regarding physiological conditions within and/or in proximity to a wearer's body, including a vital sign, biodata and temperature within an artificially created cavity (created cavity temperature or “CCT”) in a wearer's body.

### BACKGROUND OF THE INVENTION

The Quantified Self is a movement which desirably incorporates technology, communications, measurement, quantification and/or assessment of data, including self-tracking data, to improve the physical, mental and emotional health of an individual or group of individuals, as well as the wellness and daily function of an individual by use of self-tracking data. With the Quantified Self movement becoming more prevalent, consumers are continually seeking to utilize technology to acquire additional data regarding aspects of a person's daily life in terms of inputs (e.g. food consumed, quality of surrounding air), states (e.g. blood oxygen levels, body temperature), and/or performance. This self-monitoring concept combines environmental and/or wearable sensors with computing devices to produce an output that desirably improves daily functioning and allows a user to more accurately track and optimize their health.

Increased consumer interest in personal health has led to a variety of personal health monitoring devices being offered in the market. For example, wearable electronic devices for monitoring personal health are well known in the art, which include electronic measurement devices that can be worn on finger, wrist or any other body part. Generally, such devices include electronic elements, such as flexible printed circuit board(s), processor(s), sensor(s), battery(ies) and the like, with the device worn close to and/or on the surface of the skin, where they detect, analyze, and transmit information concerning e.g. body signals such as vital signs, and/or ambient data and which allow in some cases immediate biofeedback to the wearer. Also commonly referred to as wearables, fashion technology, smartwear, tech togs, streetwear tech, skin electronics or fashion electronics, wearable devices such as activity trackers are an example of the Internet of Things, since “things” such as electronics, software, sensors, and connectivity are effectors that enable objects to exchange data through the internet with a manufacturer, operator, and/or other connected devices, without requiring human intervention. Wearables are popular in consumer electronics, most commonly in the form factors of smartwatches, smart rings, and implants, including FitBit monitors (commercially available from FitBit of San Francisco, CA. USA), Apple Watches (commercially available from Apple, Inc. of Cupertino, CA, USA), Samsung gear Fit2 Bracelets (commercially available from Samsung Electronics Co. Ltd. Of Suwon, South Korea) and Oura Rings (commercially available from Oura Health Ltd. Of Oulo, Finland). Most wearable devices measure a variety of body conditions (i.e., temperature, heart rate variability, blood oxygenation levels, pulse rate, breathing rate, and so on) on a person's skin, and often perform other functions.

### SUMMARY OF THE INVENTION

At least one aspect of the present invention includes the realization of a need the art for the development of a wearable device for measuring many of a wearer's physiological conditions on a daily and/or nightly basis including, but not limited to, one or more of body temperature, heart rate at rest, heart rate during activity, heart rate variability, oxygen sensing, blood oxygen level, resting pulse rate, active pulse rate, breathing rate, movement, sleep, blood glucose levels, blood pressure and/or SpO2 sensing. In addition, the accurate and useful monitoring of an individual's body temperature during daily activities or sleeping, including changes in such temperature, is needed. Furthermore, a physiological condition monitoring device that offers constant monitoring through a convenient at-home device is desired. Such a device can represent an improvement to advance the Quantified Self movement by offering a controllable, self-tracking method for determination of a variety of health issues and/or related conditions such as fever, stress, seizures, physical activity levels, sleep and sleep patterns, women's health, menstrual health, general health, infection, virus exposure, pregnancy, perimenopause, menopause, infertility and/or fertility. Additionally, the disclosed devices and related systems can greatly benefit the public health field by identify health issues at earlier stages, potentially reducing medical cost by eliminating undiagnosed or unaddressed heath concerns, women's health conditions, menstrual health conditions, the need for excess doctor visits and/or providing alternatives to chemical-based testing procedures such as fertility determinations.

In one specific embodiment, the present invention provides a device for measuring a useful body temperature, heart rate at rest, heart rate during activity, heart rate variability, oxygen sensing, blood oxygen level, resting pulse rate, active pulse rate, breathing rate, movement, sleep and/or SpO2 sensing via an artificially created cavity within a human body, meaning a cavity in the device wearer's body that is not typically a result of natural anatomy. In humans, a wide range of potential sites are available for temperature measurement, including the skin surface, the axilla or armpit and/or the groin, as well as within existing body cavities such as the mouth, car, nasal passages, esophagus, rectum, pulmonary artery, and urethra or bladder. Superficial sites (skin, axilla, groin) are most commonly used to detect patient temperature due to their accessibility, but the accuracy of measurement from such sites are often compromised by a wide variety of variables, including perspiration, ambient air circulation humidity, use of blankets and/or peripheral vasoconstriction, among others. Deep body sites (esophagus, bladder, rectum) often more accurately reflect core body temperatures, but access to and continuous monitoring of deep body sites is much more cumbersome to use and set up, and such monitoring greatly limits a wearer's mobility and daily activities. While existing natural body cavities and/or openings can provide temperature measurements for a variety of uses, naturally existing body cavities are used for many other daily activities (i.e., eating, breathing, talking, hearing), and measurement instruments placed within such cavities typically prevent or substantially interfere with these other daily activities for the duration of the measurement. Moreover, the placement and/or presence of monitoring devices within naturally existing body cavities is typically neither comfortable nor aesthetically pleasing for the wearer for an extended period of time. The creation of an artificial cavity, however, provides for repeatable and/or continuous access to various tissues and/or tissue regions/planes positioned below the surface of the skin, including subcutaneous and/or subdermal anatomical regions having temperatures more accurately reflective of a wearer's core body temperature, and the placement of measuring devices within an artificial cavity will desirably not appreciatively interfere with the wearer's mobility and/or daily activities.

In various embodiments, the device can determine a sensed cavity temperature or created cavity temperature (CCT). Depending upon the location of the artificial cavity and the patient's unique anatomy, the CCT may be highly reflective of one or more of the core body temperature (CBT), the basal body temperature (BBT), the internal body temperature (IBT) and/or the surface body temperature (SBT), although the temperature inside of the created cavity need not necessarily correlate on a relationship basis with CBT, BBT, IBT and/or SBT. In one non-limiting example, the cavity can be an ear-piercing cavity in an earlobe (lobulus auriculae) of a human being. The human earlobe is the soft, fleshy part of the outer ear, and receives a large blood supply from the posterior auricular branch of the external carotid artery, which supply is highly reflective of the internal body temperature. The disclosed device can be similar in size, shape and/or weight to a pearl stud earring (or other ornamental type of fashion and/or functional ear jewelry), which can be worn during sleep to monitor a woman's fertility trends, where the created cavity was the result of an ear-piercing procedure which may have occurred many years prior to acquisition of the presently disclosed devices. In this example, the device is inserted into the created cavity through the two holes, an entrance and exit, originally formed by the piercing procedure. In another non-limited example, the device may be similar to a belly button stud and/or ring, nose stud/ring, eyebrow stud/ring, lip stud/ring piercing, tongue stud/ring piercing, genital stud/ring piercing or any other piercing on the body.

In various embodiments, the device can include at least one wearable sensor and/or biosensor or temperature sensor, which can be positioned, for example and without limitation, within and/or through the created cavity. The sensor may operate periodically or constantly through a specific time interval, where the device reads temperature periodically or constantly. A wide variety of temperature or other metric sensing devices are contemplated herein, including thermocouples, resistance temperature detectors (RTD's), thermistors (including negative temperature coefficient thermistors or NTC), semiconductor based integrated circuits, semiconductor sensors, silicon diodes, infrared sensors, bimetallic devices, thermometers and/or change-of-state sensors.

According to at least one exemplary embodiment, a post of the device is sized and configured to substantially or fully occupy the created cavity, and substantially close any gap between the cavity openings through which the device is inserted. If desired, the earring base and a corresponding closure device can be placed to abut against opposing ear surfaces to assist with maintaining a stable temperature measurement inside the created cavity.

Various embodiments disclosed herein desirably utilize an artificially created physiological cavity, such as a piercing cavity which extends between two adjacent skin surfaces of a human body. Within this artificial cavity, the device can include a sensor or other device which measures the CCT and/or other body conditions, with this CCT desirably corresponding to the wearer's core body temperature or other desired temperature in some manner. Primarily, the created cavity will not exist naturally with the human body-but the created cavity has rather been “added” to the wearer's body for a variety of different reasons. Of course, depending upon the device design and a wearer's natural anatomy, it is contemplated that a natural cavity or orifice might be accommodated by the device for some wearers.

In various embodiments, the devices disclosed herein can accurately measure both a temperature within the artificially created physiological cavity of a wearer as well as an external environmental temperature such as an ambient air temperature and/or water temperature (during swimming, hot tubbing, bathing, etc.), which temperature readings can be periodically and/or continuously logged and/or transmitted to a computing device such as a cell phone or computer. The provision of external temperature measurements concurrent with the sensed physiological temperature measurements can greatly enhance the accuracy and reliability of the present system as external temperature fluctuations can often influence a wide variety of patient temperature measures.

Similarly, some embodiments of the disclosed devices may include a variety of additional components and/or sensors located within, inside, adjacent or in near proximity to the wearer's artificial created cavity to detect other wearer activities, such as accelerometers which can measure wearer movements and/or wearer activity levels, GPS sensors, LED sensors, pressure sensors, or the like. Such additional measurement capabilities will desirably further improve the accuracy and/or reliability of the system by incorporating data which may be reflective of wearer events or happenings that may influence the accuracy and/or reliability of the sensed internal temperature measurements, including providing the system with an ability to detect, analyze, highlight and/or discount various data points or trends (including anomalous and/or unexpected readings) that the system may detect-especially where artificial intelligence (AI) systems may be utilized in the analysis of the resulting wearer data.

In various embodiments, the wearable device can further contain a miniaturized transmitter/receiver with optional wireless communication components. To power these components, one or more batteries can be incorporated into the wearable device, with miniaturized low power rechargeable battery components potentially employed to provide power to various portions of the system.

Desirably, the system will be small, compact and lightweight in design, for example by using various combinations of rigid and/or soft or flexible materials in the design, thereby increasing the comfort and case for wearing at home during sleep and during daily activities.

It is contemplated that, in operation, the system is used for measurement of CCT and/or other anatomical information, with information available for tracking and/or deriving medical useful information and determining fertility, menstrual cycle health, blood pressure, heart health, breathing and lung health, fever, sickness, acute pain, chronic conditions or constant temperature and vital sign and biodata monitoring system for hospital use. A wide variety of uses and environments for the disclosed system components are contemplated herein, including use of the device for: Fertility, Infertility, Natural Family Planning, COVID, Infectious Disease, Cancer, Long term hospital care, Fever, Thyroid Issues, Gynecological Health, Pregnancy, Material health conditions, Gut Health, Diabetes, Obesity, Asthma, Premenstrual syndrome, Polycystic ovary syndrome, Endometriosis, perimenopause, menopause, Osteoporosis, Menstrual disorder, Urinary tract infection, Heart Disease, eclampsia, Chronic Obstructive Pulmonary Disease, Women's Health, Wellness Management, Stress, Sleep, Diet/Calorie Counting, Activity/Fitness, Sports, Government Health, Military, Agriculture, etc.

### BRIEF DESCRIPTION OF THE DRAWINGS

A full and enabling disclosure, including the best mode thereof, to one of ordinary skill in the art, is set forth more particularly in the remainder of the specification, including reference to the accompanying Figures. The Figures illustrate certain aspects of the current invention and together with the description, serve to explain, without limitation, the principles of the invention. Like reference characters used therein indicate like parts throughout the several drawings.

FIG. 1 depicts an exploded perspective view of one exemplary embodiment of a wearable sensing device in the form and shape of a post-type earring:

FIG. 2 depicts the assembled wearable sensing device of FIG. 1:

FIGS. 3A through 3F depict various views of a flex board and battery of the wearable sensing device of FIG. 1:

FIG. 4 depicts an exemplary layer cross-section of the flex board FIGS. 3A and 3B:

FIG. 5A depicts a first surface and component layout of the flex board of FIGS. 3A and 3B;

FIG. 5B depicts a second surface and component layout of the flex board of FIGS. 3A and 3B

FIGS. 6A through 6E depict pin hole positions within the various layers of the flex board of FIGS. 3A and 3B from top to bottom:

FIGS. 7A through 7F depict exemplary mask layers in the flex board of FIGS. 3A and 3B from top to bottom:

FIGS. 8A through 8C depict exemplary circuit diagrams for the wearable sensing device of FIG. 1:

FIG. 9 depicts a sensor positioned within the stud post assembly of the wearable sensing device of FIG. 1:

FIGS. 10A through 10C depict various views of one exemplary embodiment of a charging and storage case for the wearable sensing device of FIG. 1:

FIGS. 10D and 10E depict views of an alternative embodiment of a charging and storage case for an earring device.

FIGS. 11A through 11D depict various graphical user interface displays (GUI's) for exemplary embodiments of an application program or APP loaded on a mobile phone:

FIG. 12 illustrates an exemplary flow chart of data processing applied to CCT measurements or values by the Application:

FIG. 13 graphically illustrates an exemplary use of temperature measurements in relation to time for determination of fertility:

FIG. 14 an alternative embodiment of a wearable sensor device which can be used as a closure, backing or similar attachment device to a multiplicity of earrings;

FIG. 15A depicts a series of existing earrings prior to securement using the wearable sensor device of FIG. 14:

FIG. 15B depicts advancement of the wearable sensor device over an earring post, and

FIG. 15C depicts the wearable sensor device secured to an earring post with a sensing device within the piercing cavity of a human ear.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure cover such modifications and variations as come within the scope of the appended claims and their equivalents.

Ranges can be expressed herein as from “about” one particular value, and/or to “about” another particular value. When such a range is expressed, another aspect comprises from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description comprises instances where said event or circumstance occurs and instances where it does not.

The present disclosure also incorporates the disclosure of U.S. Pat. No. 10,117,643 entitled “Created Cavity Temperature Sensor,” filed Apr. 25, 2015, which is hereby expressly incorporated by reference herein. The components disclosed herein may be utilized in systems and/or methods for monitoring characteristics of humans and other animals, including those inventions and concepts disclosed and described in U.S. Pat. No. 10,117,643 to Gevaert et al.

The present disclosure is generally directed towards a device to measure various physiological conditions of a wearer. Such measurements can include sensing a useful body temperature and/or variations thereof over time, heart rate, breathing rate, blood oxygenation, pulse, movement, activity, calories, distance traveled, steps, blood pressure, glucose monitoring, angular velocity measurements, location (e.g. global positioning), magnetic field measurements, and ambient noises and other conditions.

More specifically, among other things the device is capable of measuring the created cavity temperature (CCT) within an artificial created cavity in living tissue or the wearer's body into which the device is inserted. This current device provides a wearable thermometer that continuously or periodically measures temperature for a convenient, comfortable method of continuously tracking the wearer's temperature. Alternative or in combination, the device may include LED sensors for heart rate, breathing rate, blood oxygenation and/or pulse measurements. Alternative or in combination, the device may include an accelerometer for movement, activity, calories, distance traveled and/or steps. Alternative or in combination, the device may include LED sensors and/or Pressure sensors for blood pressure. Alternative or in combination, the device may include a CGM sensor for glucose monitoring. Alternative or in combination, the device may include a gyroscope for angular velocity measurements. Alternative or in combination, the device may include a global positioning system sensor. Alternative or in combination, the device may include a magnetometer for magnetic measurements. Alternative or in combination, the device may include a microphone and/or speaker(s) for ambient environment monitoring, or for use listening to music, responding to phone calls, etc.

### Created Body Cavity

It should be understood that the disclosed device(s) can be utilized in a variety of locations on/in a human body, although for many individuals a body piercing location such as one or both ear lobes may be particular preferred (and such locations may already be pierced to accept a variety of ornamentation). Various piercing locations may be suitable for biometric measurement and sensing of temperature, heart rate at rest, heart rate during activity, heart rate variability, heart beat sensing, oxygen sensing, blood oxygen level, blood pressure, resting pulse rate, active pulse rate, perfusion, breathing rate, movement, sleep, SpO2, and/or glucose, measurements of bacteria, white blood cell count, proteins, lipids, salts, fats (or other fluid characteristics, including lymph or other fluid characteristics) as well as a variety of other data including location (via GPS), sound sensors, accelerometer data, etc. In some cases, localized body conditions proximate to a specific piercing location may be particularly well suited for measurement of various physiological conditions, such as a tongue piercing for tracking glucose measurement and/or blood sugar levels.

The disclosed CCT is a measurement that may be obtained from an artificially created cavity in the wearer's body, where the created cavity is formed artificially, as in a non-limiting example, an earring piercing procedure. Additionally, two or more artificially created cavities can be utilized for measuring CCT in one body. As one non-limiting example, two different biosensing devices or wearable sensing devices can be used in two different created cavities to measure two CCT values (or other anatomical metrics) simultaneously within separate created cavities. Further, these different sensing devices can be used to derive a single CCT (or other anatomical metrics). It should be understood that the CCT (or other anatomical metrics) from one created cavity in a wearer's body may by different from the CCT (or other anatomical metrics) obtained from another created cavity in the same wearer's body, as anatomical differences may induce localize temperature variations (e.g., the CCT or other metric of an ear piercing may not be the same as the CCT or other metric of a belly button or tongue piercing of the same wearer, or the CCTs or other metrics in opposing ear piercings may be different for a variety of environment and/or anatomical reasons).

The disclosed device can desirably measure CCT or other metrics within the created cavity to track the wearer's useful temperature or for other purposes. The aspects of the created cavity are further described below. CCT may or may not be the same temperature measurement as core body temperature (CBT), internal body temperature (IBT), basal body temperature (BBT) body cavity temperature (BCT), and/or surface body temperature (SBT). This can be recognized by reason that the temperature inside the created cavity does not need to equal and/or correlate on a relationship basis with CBT, IBT, BBT, BCT and/or SBT. Moreover, it is suitable if CCT measurements are a perfect match, higher, lower, or not a 1:1 relationship to body temperature.

The disclosed device can offer a practical design that provides means for a comfortable, convenient and unobtrusive method of continuously or periodically tracking the wearer's temperature or other biometrics while sleeping, rest or activity. For example, but not limited to, the shape of the device resembles a small, stud earring structure that is wearable on the body, through a wearer's earlobe, wearer's belly button, tongue, nose, eyebrow, lip, genitalia and/or other body locations. This device allows for wireless communication to an external system for tracking continuous or periodic measurements, which can be used for the non-limiting example of determination of fertility by identifying changes in body temperature associated with the biological event of ovulation.

By means of the disclosed device, cavity temperature measurements and/or other measurements are obtainable from a wearer over a defined period of time. In a non-limiting example, cavity temperature measurements can help assist in the determination of fertility. Within this example, readings are evaluated to differentiate the local minimum during a given night's sleep, where the local minimum is defined by the lowest reading within a pre-established time period. Then local minima can be plotted in relation to time for fertility trends. Additionally, measurements can be tracked over 24 hours and temperatures compared across different days and nights to determine patterns, for a non-limiting example, to identify or predict the wear's date of ovulation.

### Ovulation and Fertility Detection

12.3 percent of women (7.5 million) in the United States ages 15-44 have impaired ability getting or staying pregnant, according to the Centers for Disease Control and Prevention (CDC). In 40% of such women, ovulatory defects are present but difficult to characterize because ovulation is an internal, normally unmonitored clinical state that evolves quickly over short time frames. Defining the rapidly changing physiology of ovulation for each affected woman requires frequent monitoring during suspected ovulation windows. Moreover, while the rhythm method of birth control (e.g., tracking a menstrual cycle on a calendar to predict ovulation) is over 75% effective at preventing unwanted pregnancy, less than 1% of women 15 to 44 years of age currently use this highly effective natural form of birth control.

In predicting ovulation for both pregnancy and birth control objectives, the need for frequent monitoring currently translates to significant patient burdens. The use of basal body temperature (BBT) recordings is a known and safe method to monitor ovulation and has the advantages of patient monitoring themselves at home (no clinical scheduling or attendance) at minimal cost and risk compared to a blood tests or ultrasounds. However, application of BBT methods is limited by the inconvenience of taking and recording a waking temperature at the same time each morning-patient compliance is especially poor. Even for those who comply, the information is difficult to interpret and often frustrating for the patient.

Traditional fertility thermometers assist in tracking a woman's ovulation trends by measuring her basal body temperature (BBT) through a natural body cavity. These devices do not offer the most accurate ovulation results due to temperature measurements taken after awakening with a non-convenient thermometer. In contrast, the disclosed devices and associated system components will desirably eliminate any need to awaken before temperature measurements can be taken and more accurately identify the low temperature within a given night's sleep, because the low temperature does not necessarily occur at the time of waking. This is desirably accomplished by a small, wearable sensor such as a temperature sensor located within an artificial created cavity in the body that offers continuous or periodic readings of CCT that are wirelessly transmitted and analyzed by the wearer's associated smart device.

In one exemplary embodiment, the disclosed earring technology and systems conveniently monitors ovulation by measuring basal body temperature (BBT) utilizing earring sensors-similar in visual appearance to the earrings that many women in the US wear daily. Since the monitoring occurs at home, the inconvenience and cost to the patient is mitigated and care can be delivered more equitably. Moreover, the applications of this disclosed device can go far beyond infertility and can extend to use in identifying ovulation/avoiding pregnancy in fertility management, assist with wellness tracking, perform infection/COVID monitoring and/or other uses, provide early pregnancy detection, assist with natural birth control, identify pregnancy, labor and menopause onset, and/or rack a wearer's menstrual cycle and/or other health metrics. In various embodiments, the earring may include features which may alert the wearer (i.e., using sound, vibration, light and/or electrical pulses to the user) of vital information, such as device proximity or non-proximity (i.e., phone theft prevention) or the receipt of emails, text messages, information updates or phone calls, etc. The disclosed devices and related system components also provide capabilities to obtain information based on early detection of one's health issues or conditions, like infection, COVID19, virus, pregnancy, early labor, perimenopause, women specific conditions or needing to take specific medications or vitamins. This device could also be used for other medical applications, by way of non-limiting example as seen in detection of fever and/or sickness. Further, this device can be used for deriving information for medical tracking and implemented for hospital use in circumstances that require constant or periodic temperature and vital and biometric monitoring. Additional applications contemplated herein include, but are not limited to, fertility, infertility, natural family planning, COVID infection and/or condition, infectious disease symptoms and/or susceptibility, cancer diagnoses and treatment, long term hospital patient monitoring, fever detection and treatment, thyroid issues, gut health, diabetes detection and management, obesity, asthma, heart disease, chronic obstructive pulmonary disease, women's health issues, wellness management, stress management, sleep monitoring and disorder detection and treatment, diet and calorie counting, activity/fitness tracking, sports, government health, military, agriculture, etc.

### Device Design and Dimensions

The present invention includes a multiplicity of individual components that are assembled into an earring or other piercing device that can be, in a non-limiting example, approximately ½ inch×½ inch, preferably ¾ inch×¼ inch most preferably ½ inch×¼ inch in total size, although a wide variety of device sizes, shapes, weights and/or designs are contemplated by the present disclosure. In general, the device will desirably be sized and/or shaped to comfortably fit into and/or through a created cavity such as an ear-piercing cavity (or other piercing cavity) while accommodating the surrounding anatomy of the wearer such as adjacent skin surfaces, ear lobes, etc. In addition, the weight of the device will desirably be minimized where possible to reduce stress and/or damage to relevant body parts in which it resides, such as the ear lobes, etc.

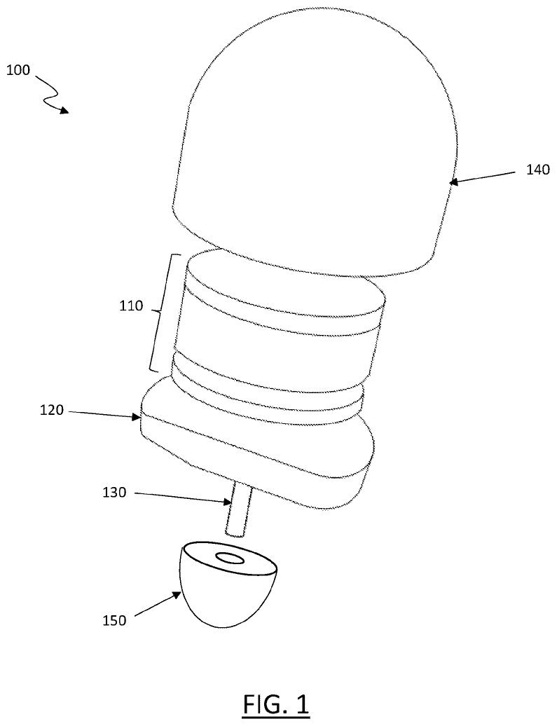

FIG. 1 depicts a simplified exploded perspective view of a body-mounted sensing device in the form and shape of a post-type earring 100. The earring 100 includes a central body 110, a base 120, a post 130, a surface dome or cover 140 and a clasp or backing 150.

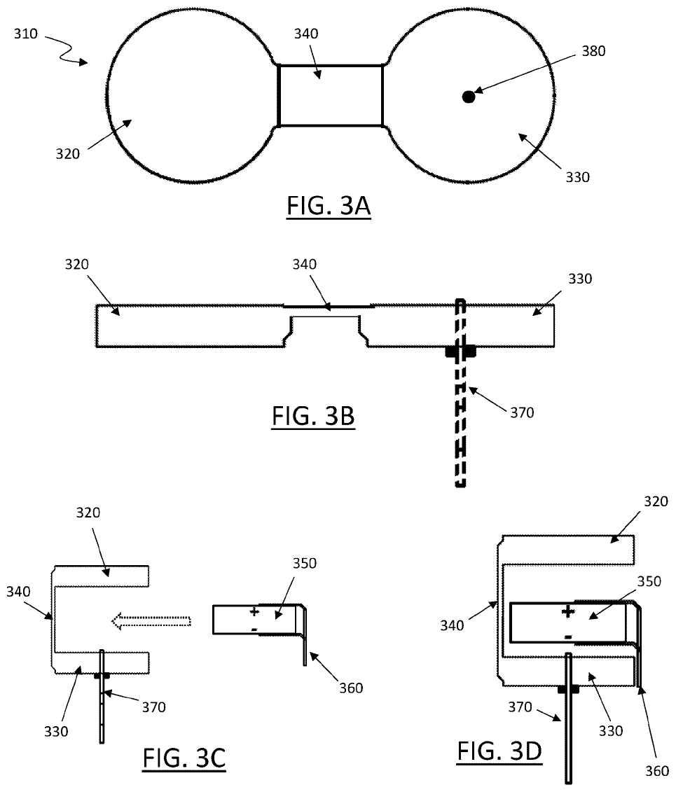

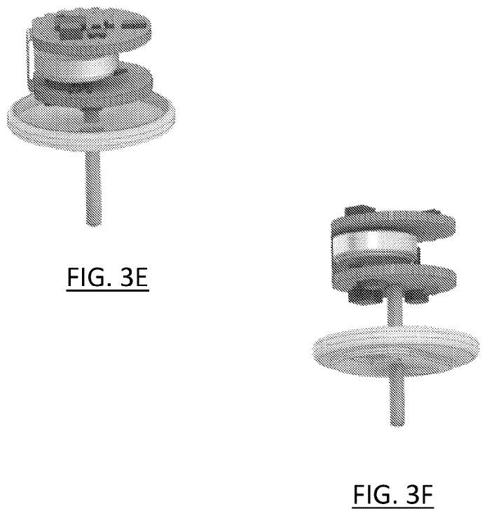

As best seen in FIGS. 3A through 3D, the central body 300 can include a semi-flex board, flex board or flexible circuit board 310 having a first rigid section 320, a second rigid section 330, and a flexible linkage 340 positioned therebetween and connecting the two rigid sections. The shape of the two rigid sections is desirably round to fit within the dome enclosure. Exemplary dimensions for the flexboard components can include a thickness of 825 μm (±120 μm) and a diameter of approximately 10 mm for each rigid section, with a 133 μm thickness (+50 μm) for a 6 mm long flexible section having a width of 3.48 mm and a 0.55 mm radius of flex.

In the depicted embodiment, a power supply or battery 350 can be positioned or sandwiched between the first and second rigid sections for sizing considerations and/or a variety of other reasons, although a wide variety of component configurations and/or arrangements can be utilized by those of ordinary skill in the art of circuit design. In various embodiments, the disclosed devices desirably utilize minimal power and have the capability to measure a wearer's temperature or other vital signs or biodata via the earring post within the ear piercing (and/or other measurements) at least every 15 mins for a minimum of eight (8) hours before requiring recharging. More preferably, the disclosed devices allow for the capability to measure a wearer's temperature via the earring post within the ear piercing every 15 seconds for a minimum of twenty-four (24) hours before requiring recharging. As described herein, the carrying and/or storage case for the earrings may incorporate battery charging features which are integrated into the portable storage case.

Various types of batteries may be utilized for any of the components described herein, including films, flex, rechargeable, non-chargeable, electronic charging, solar-power charging, trickle charging, battery maintainers etc.

FIGS. 3C and 3D depict the exemplary flexboard 310 of FIGS. 3A and 3B after being flexed to a desired “stacked” configuration, with a battery 350 (e.g., a 3.7 Volt 13 m-A-hr Li-ION battery commercially available from Shenzhen Grepow Battery Co., Ltd. of Shenzhen, China) desirably positioned between the rigid sections 320 and 330 with battery leads 360 shown extending along and/or connected to ports on the second rigid section 330. The first rigid section will desirably house the MCU, memory and RF antenna, with this section preferably thermally isolated to some degree from the more heat generating second rigid section, as well as isolating the digital signals from the analog signals. The second rigid section will desirably house the power, analog sensors, post assembly and battery (which can be soldered as a post process of SMD placements). The battery leads are desirably soldered to castellated edge connections on this section. The first rigid section can also incorporate castellated edge connections for SWD Debug/Programming Interface between MCU and External Debugger/Programmer.

In various embodiments, a miniature transmitter is included on the flexboard and is used for interfacing the wearable sensor, LED sensors, accelerometer, and other sensors to a measurement tracking or control device. The transmitter can be positioned on an end of the device and is desirably located outside of the artificial created cavity. The transmitter contains the capability to isolate, amplify, filter noise, linearize, and convert input signals from the data sensors and wearable sensor and send a standardized output signal to the computing/control device. Common electrical output signals ranges are used.

As best seen in FIGS. 3B through 3D, a central post 370 will desirably extend through and be secured within an opening in the second rigid section 330. In a preferred embodiment, an overall dimension of the earring device can be an outer diameter of 12 to 13 mm, with the dome shaped and/or colored to present a pearl-like appearance or other desired colors or styles. In various alternative embodiments, the device can have a width/diameter of approximately less than 16 mm, and more preferably less than 12 mm. The post can have a length of approximately 6 to 8 mm and a diameter of approximately 1 mm, although other posts having lengths of 4.5 mm, 6 mm, 8 mm and 10 mm and diameters/post thicknesses such as 0.8 mm, and 1.2 mm (and/or other sized known in the art) are contemplated herein with various design changes. The earrings can each have a total weight of less than 8 grams, and more preferably less than 5 grams, and desirably incorporates a comfortable and lightweight design and outer profile to allow the user to wear the earrings during sleep.

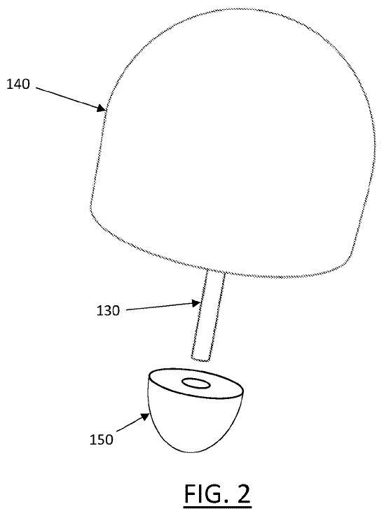

As best seen in FIGS. 1 and 2, the base component 120 will desirably fit partially and/or fully within the cover 140 (e.g., preferably via seamless outer and inner molding), with a lower surface of the base positioned adjacent to or against a skin surface of the wearer (e.g., an ear surface for an earring embodiment) when the post is contained within the piercing channel. In various embodiments, the device components will desirably be fully sealed and capable of full water immersion, including during bathing and/or showers. In some embodiments, additional water protections may be provided, such as 1 meter, 3 meter and/or 100 meter water proof/water resistance measures.

For one non-limiting example, the cover component can comprise a small, spherical housing, approximately ¼ inch diameter, containing all electrical components needed to operate the wearable sensors and/or biosensors and/or temperature sensors and/or fluid sensors and associated components. Such a design can desirably emulate the profile, shape and/or coloration of a small, pearl earring or similar design.

While the disclosed embodiment is a one-piece earring, it is contemplated that an alternative design could incorporate a two-piece construction, such as where the earring device comprised two parts, including a proximal portion that is able to be disconnected from a distal portion. For example, the distal portion could comprise a miniature transmitter, other components and/or at least one wearable sensor and biosensor and temperature sensor, while the proximal removable portion could comprise a miniature battery. The proximal portion can be connected to the distal portion by means of the post (e.g., containing a temperature sensor and/or other biosensors and/or wearable sensors and/or microphone and/or speaker), with the proximal portion encompassing the capability to detach by manually sliding off the post, similar to an earring back. By removing the proximal portion of the device, inserting the distal portion of the device through a created cavity, and returning the proximal portion onto the device to provide energy for the various components thereof, CCT or other biometrics can be measured and tracked.

In various embodiments, as shown in FIG. 1, the post 130 and/or base 120 can desirably incorporate open and/or clear/transparent portions which allow various components to access the skin surface and/or transmit/receive information from the wearer's anatomy, such as LED transmitters and/or sensors to detect and/or calculate wearer anatomical measurements such as heart rate, pulse, breathing rate, blood oxygenation and/or CO2 levels. A variety of sensor types can be incorporated into the device, including a wide variety of biochemical (enzyme-based, tissue-based, immunosensors, DNA biosensors, and thermal and piezoelectric biosensors), chemical, electromechanical, optical and/or electrical sensors. For example, a chemical sensor may be included to measure the concentration levels of chemicals in blood, sweat or other bodily fluids, such as glucose monitors for diabetics and/or lactate level measurements, as well as sensors to measure proteins or hormones, fertility hormones or other chemical constituents (e.g., stress hormones) in sweat. Similarly, an electromechanical sensor can be incorporated to use electrical measurements to track mechanical movements, such as an accelerometer to measure physical activity and/or device/wearer orientation, inertial measurement units to measure angular changes and/or linear acceleration (e.g., for rotational velocity and/or position tracking) or GPS. Optical sensors can be incorporated to detect various biological signals like heart rate, heart rate variability, pulse, breathing rate, oxygen saturation and/or blood pressure (as well as temperature, galvanic skin response and/or stress sensors), with these sensors typically including a light source and photodiode sensors that measure how much light is absorbed, reflected back out and/or passed through adjacent tissues via spectroscopy analysis. Electrical sensors (including bioelectrical sensors and electrochemical sensors) can be included to detect, measure and evaluate electrical signals in the wearers tissues, including to measure heart rate or brain activity, including electrocardiogram information (e.g., ECG or heart rate monitor), EEG measurements (electroencephalograms) electromyography measurements (EMG or muscle movement monitor) and/or electrode dermal sensors to measure sweat levels (e.g., perspiration monitoring). Other sensors contemplated herein include pressure sensors, CGM sensors, Gyroscopes, GPS receivers and other wearable sensors.

In various embodiments, combinations of the following measurements and/or sensors are contemplated (including in any combinations thereof): Temperature, Pulse, Resting Heart Rate, Heart Rate Variability (HRV), Heart Beat Sensor, Perfusion, Oxygen Level, Blood Oxygen Level (SpO2), Breathing Rate, Blood Pressure, Glucose, Hormones, GPS, Accelerometer, Motion Sensors, ambient and/or cavity microphones and speakers.

Where a measurement sensor or wearable sensor, for a non-limiting example wearable sensor is incorporated into the device, this is desirably a small linear or non-linear rod-like structure, located within and/or on a structure that passes into or through the created artificial cavity, with the sensor in electronic communication with a circuit board containing operational software for the sensor. The wearable sensor can desirably sense or measure temperature or temperature changes constantly or periodically using specific or nonspecific time intervals. In one non-limiting example, this sensor functions to accurately detect small temperature changes, for measurement orders of about 1 to 0.01 degrees Fahrenheit. The temperature can be a unitary sensor unit, or a plurality of sensors can be used.

In one exemplary embodiment disclosed herein, shown in FIG. 1, the earring 100 desirably incorporates a plurality of wearable sensors, including at least one temperature sensor (e.g., thermistor sensor SC30F103AN, commercially available from Amphenol Thermometrics, Inc. of St. Marys. PA, USA) positioned within the post 130, an ambient temperature sensor (e.g., NTC thermister NCP03XH103JOSRL commercially available from Murata Electronics North America, inc. of Smyrna, GA USA) on the central body 110, an accelerometer (e.g., accelerometer MC3635 commercially available from Memsic Semiconductor Co., Ltd. of Zhubei City, Hsinchu County, Taiwan), and an optical sensor package which incorporates an optical biosensor with proximity sensor and ambient light sensing features (Renesas OB1203SD-C4, commercially available from Renesas Electronics Corporation of Tokyo, Japan). The Renasas photoplethysmography (PPG) biosensor integrates light sources and drivers, analog digital conversion and I2C communication in a single optical package, with data from the OB1203 biosensor potentially being used to determine heart rate (HR), oxygen saturation (SpO2), respiration rate (RR), pulse and/or heart rate variability (HRV—a measure of stress). In various embodiments, this device can desirably measure one or more of the following: heart rate, heart rate variability, oxygen saturation, respiration rate, 3-axis accelerometer, ear lobe cavity temperature and/or ambient temperature.

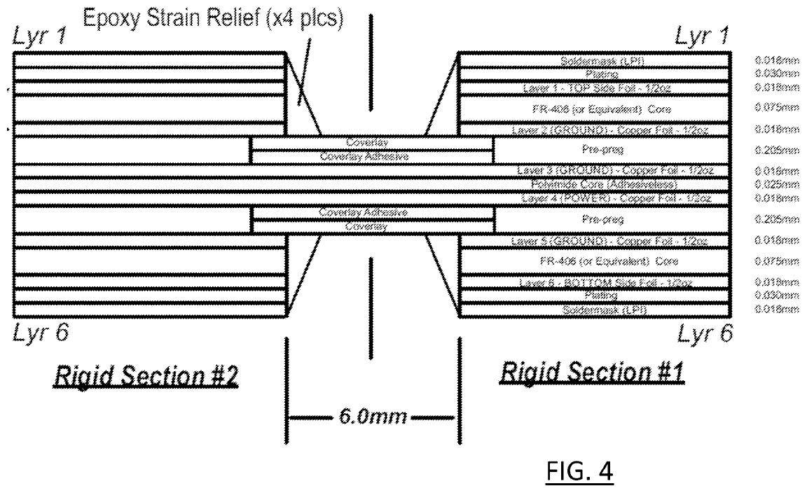

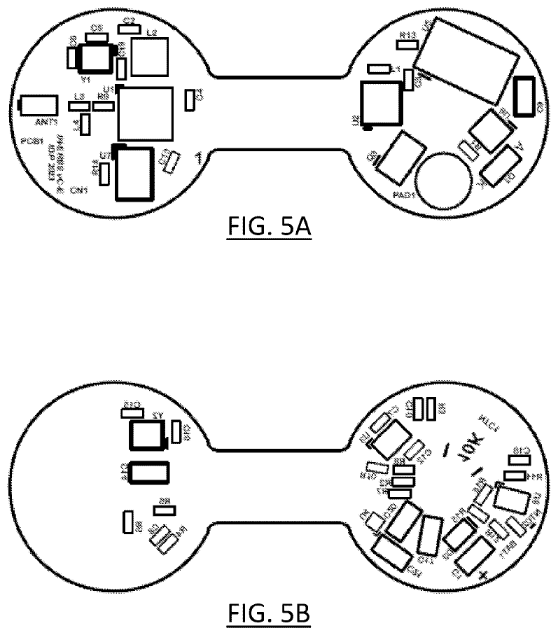

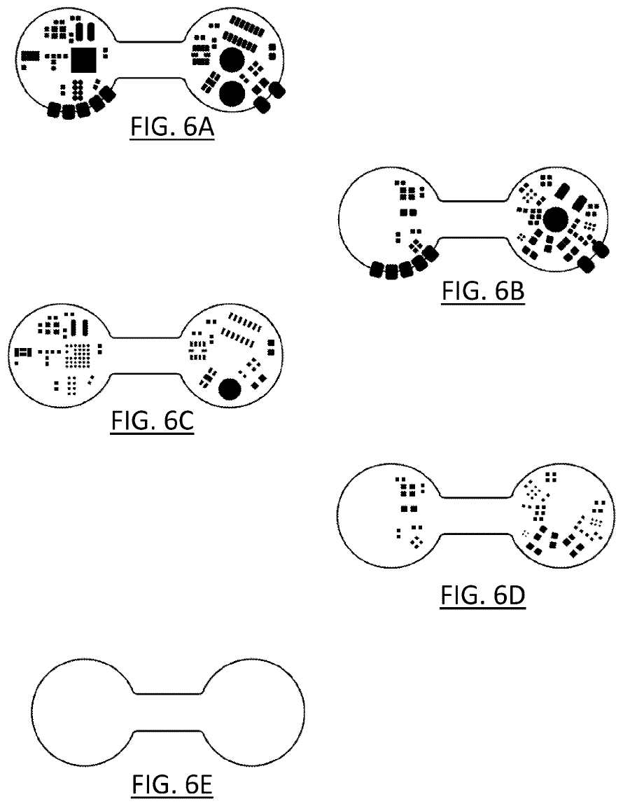

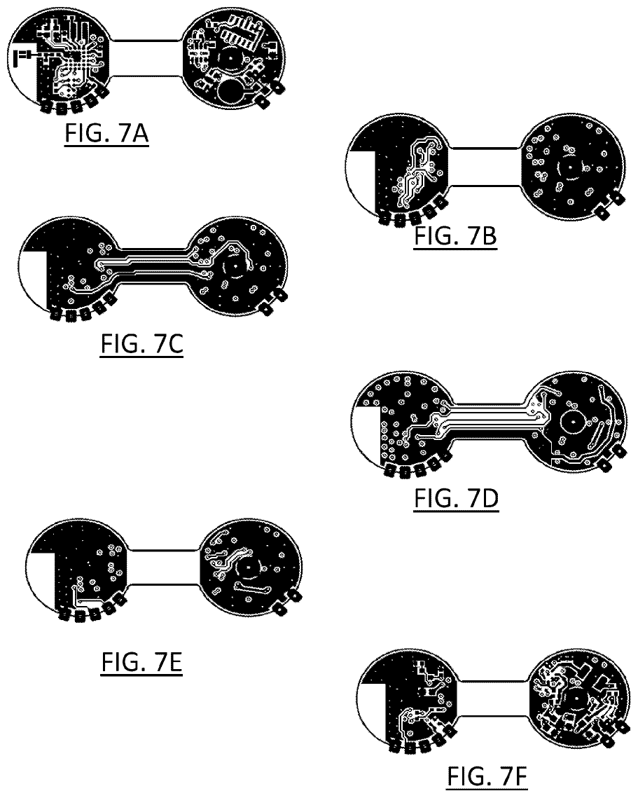

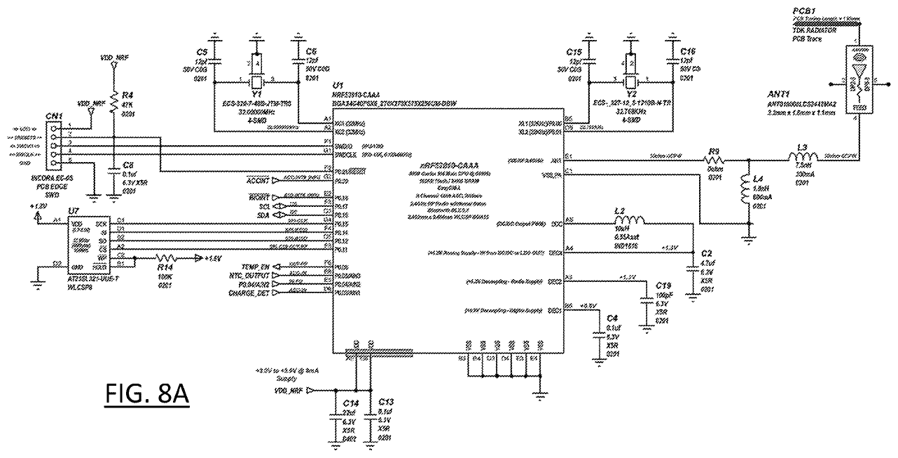

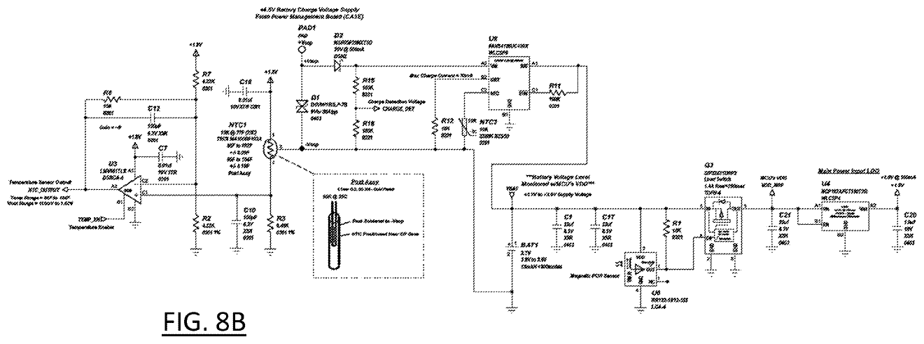

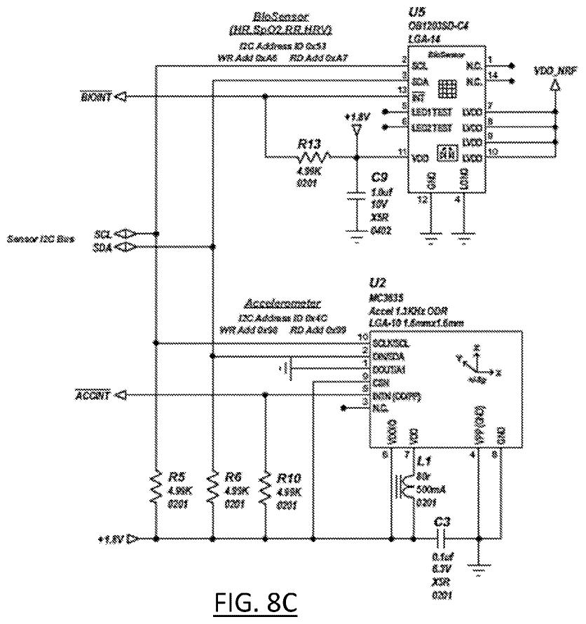

FIG. 4 depicts an exemplary layer cross-section of the flexboard 310 of FIGS. 3A and 3B, showing the various layers within the rigid and flexible section thereon. FIGS. 5A and 5B depict exemplary component positioning and placement on the upper surface of the first rigid section (FIG. 5A) and on the lower surface of the second rigid section (FIG. 5B). FIGS. 6A through 6E depict pin hole positions within the various layers of the flexboard from top to bottom, and FIGS. 7A through 7F depict exemplary mask layers in the flexboard from top to bottom. FIGS. 8A through 8C depict circuit diagrams for the exemplary device of FIGS. 1 and 2.

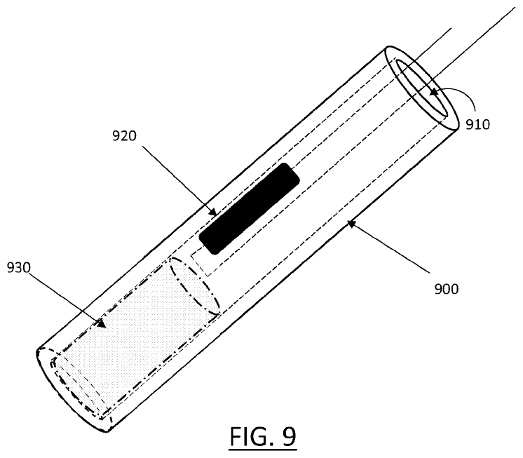

FIG. 9 depicts a partial perspective view of an earring post 900 having a hollow interior 910, with an NTC thermistor 920 (or similar metric sensing device) secured therein. To allow the selected thermistor to fit within the post tubing, this tubing will desirably have an inside diameter of 0.8 mm and an outside tubing diameter of approximately 1.1 mm to 1.15 mm, which should fit comfortably but snugly into an average earlobe piercing. If desired, a thermally conductive sealant or epoxy 930 can be placed within the tube, desirably sealing the tube end and optionally securing the thermistor against the inside wall(s) of the post. This arrangement desirably seals the cavity closed around the post, and provides good thermal transfer from the earlobe to the surface of the tubing.

The post can comprise a wide variety of materials and/or coatings thereupon, with the material desirably comprising a non-allergic metal (or other material including polymers and/or ceramics) possessing sufficient mechanical rigidity to resist bending and/or collapse during normal wear, as well as allow high thermal conduction of body temperature and the ability to be soldered to wiring and/or direct mounting onto a PCB. In the disclosed embodiment, the tubing material is stainless-steel hypodermic tubing which can be gold plated for solderability and to present a good visual appearance. The post assembly can have an outside or distal end (e.g., the end passing through the cavity away from the earring assembly) which is desirably closed and smoothed for easy, comfortable insertion of the post into and through the earlobe, and also to minimize or prevent any intrusion of contaminants. Desired component materials can include durable medical grade materials, including but not limited to nickel free metals, plastics and/or 3D printed resins, Various embodiments of the disclosed device are desirably durable, impact resistant and/or waterproof, and are suitable for long-lasting use and operation. The materials and electronic components will provide strength and capability for continuous wear during the wearer's normal daily activities.

If desired, the post assembly can be attached to a PCB's grounding plane by soldering an open end of the tubing to a PCB through-hole, which can act as a ground return for the earring recharging circuit.

Desirably the thermistor 920 will be positioned in direct or close contact with the wall of the post, with the thermistor desirably positioned within a portion of the post that extends out of the device and that will be placed within the earring cavity. As depicted, the chosen thermistor has a diameter of approximately 0.76 mm, with the small gauge wires from the sensor desirably extending out of the open end of the tubing for soldering to the PCB.

In many instances it would be highly desirable to incorporate multiple different sensing components in a single wearable device. For example, the usefulness of the temperature sensing component located within the created cavity can be greatly enhanced by the incorporation of a LED light and photosensor components in a location proximate to the skin surface of the wearer's ear-such as where the base of the device abuts against the ear skin surface. The data obtained from such combination sensing devices can be highly useful during analysis and/or assessment of the data, as conclusions obtained from one data stream might be modified and/or refuted by a second data stream and/or reinforced by a third data stream, as so on. The employment of multiple sensor types in a single device is thus contemplated by the present invention, including the use of surface sensors of one type in combination with more “invasive” sensors of a second type—such as devices resident in a created cavity. In various embodiments, it is desirable to incorporate multiple wearable sensing components into an individual earring device, including in various embodiments a temperature sensor within the post as previously discussed. Desirably, the post sensor can comprise a NTC Thermistor type for which provides for fast response time, with an R-T curve centered around the normal body temperature range of 95 F to 102 F with a preferred tolerance of +0.1 degree F. As best seen in FIG. 9, the sensor can be positioned within the stud post assembly, such that the sensor is centered and/or otherwise contained within the thickness of the earlobe cavity while being worn (e.g., between the first and second dermal skin layers at the opposing cavity openings).

For a normal sized adult (e.g., a North American individual), the average earlobe thickness is approximately 3 to 6 mm, thus the length of the thermistor within the post tubing for such an individual will desirably be approximately 3 to 4 mm within the tube (as measured from the base surface). In some embodiments, the thermistor will be positioned within the post assembly at a location just outside of the earring dome and/or base, which desirably ensures that the sensor will be positioned inside the earlobe piercing and not extend outside and/or behind the earlobe while being worn.

During use, the disclosed device is inserted through an artificially created cavity, which provides an area for the useful wearable sensor measurement and biometric measurement, for example at least one temperature measurement of CCT, heart rate, heart rate variability, blood oxygenation, breathing rate, pulse, glucose, blood pressure, hormone and other biosensor measurement means. The CCT of the created cavity can be measured by the partial or complete closure of a space between the tissue by the post and/or wearable sensor and/or temperature sensor. The cavity desirably has at least an entry hole, and in most typical piercings are also accompanied by an exit hole (although the current device can easily be used in blind cavities of a single-opening piercing). The created cavity can be a linear or non-linear cavity, as in the non-limiting example of a narrow non-linear tunnel, i.e., a belly button piercing. A piercing cavity desirably creates a small, bounded area, a non-limiting example of about 1 mm radius and 5 to 8 mm length, through the wearer's body for temperature measurements of CCT.

### Charging and Storage Case

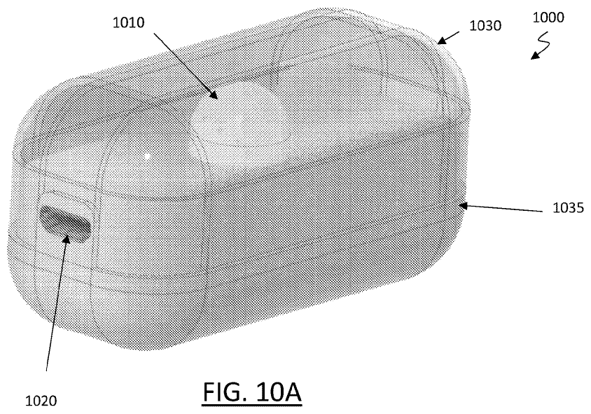

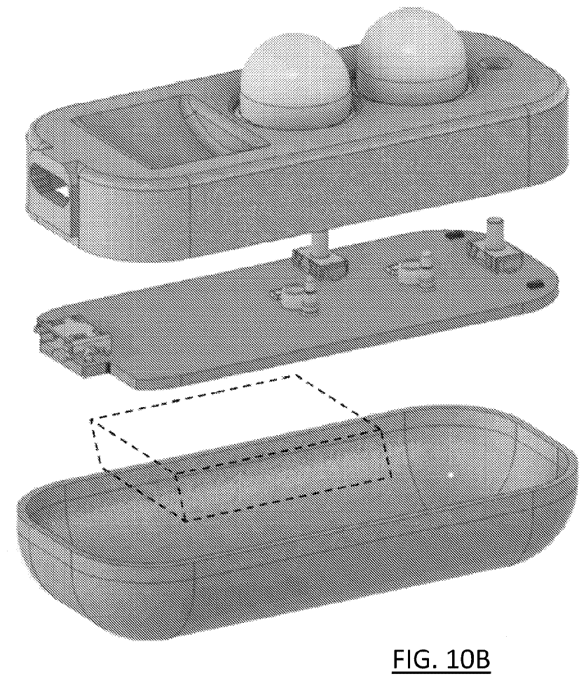

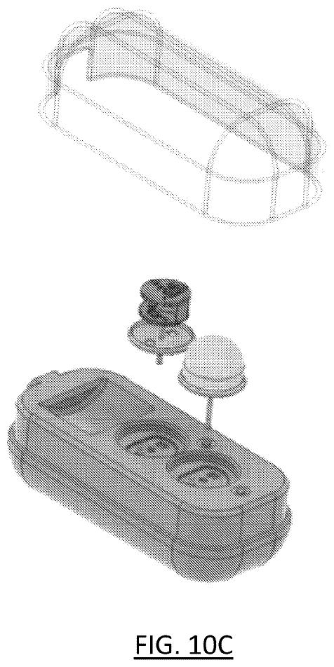

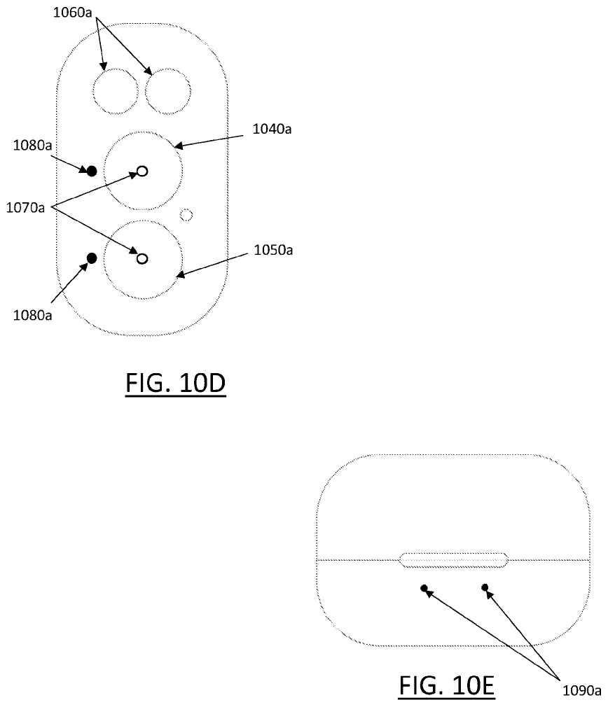

FIGS. 10A through 10C depict various views of one exemplary embodiment of a charging and storage case 1000 for an earring device 1010 (or pair thereof) such as described herein. The case can comprise a wide variety of materials, including polycarbonate and/or polyurethane with a lacquer casing (similar to the soft finish clinic white used with current AirPod (R) devices (commercially available from Apple Inc. of Cupertino, Ca USA). Other suitable case materials could include Acrylonitrile Styrene Acrylate (ASA) or Acrylonitrile Butadiene Styrene (ABS), as well as other materials known to those skilled in the art. In another preferred embodiment, the charging case resembles a jewelry box or earring case. Desirably, the case will include a charging port 1020 for connection to a power supply, such as a PCB USB Connector Type-C. In the disclosed embodiment, the case can include a hinge or flip-type lid 1030 and magnetically attached base 1035.

FIGS. 10D and 10E depict views of an embodiment of a charging and storage case for an earring device, which when opened exposes a left earring well 1040a, a right earring well 1050a and one or more auxiliary wells 1060a or depressions for clasps, backings or other securement devices. An opening 1070a is provided at the center of each of the left and right earring wells 1040a and 1050a, with each opening 1070a desirably accommodating a corresponding earring post, which desirably provides securement and charging of the earring when placed into the case. A pair of LED indicators 1080a can be provided which indicate charge and/or charging status for each of the earrings, and similar charge status or other indicators 1090a may be provided on an external surface of the case to provide earring charge information to a user without requiring the user to open the lid to obtain such information. In the disclosed embodiments, once the earrings are fully charged, the LED lights can turn off or change color. In one exemplary embodiment, percentage of battery charge or battery life or battery duration is displayed on or within the charging case. In another exemplary embodiment, percentage of battery life is transmitted and displayed on the remote software and/or app.

In at least one exemplary embodiment, the storage case can incorporate 4 LEDs, indicating the following: D1 charging status of internal battery in charging base, D3 LED indicating if battery in charging base has stored power (i.e., earrings can be charged from case without case plugged into wall), and D4/D5 are LED indicating charging of each earring. Additionally, the charging case incorporates a reset button, “power on” reset. If desired, the D4/D5 components can be alternatively used to power on reset of the earrings during MCU hangups or system restart. In the current embodiment, the third and fourth LEDs can be used to display individual Earring Charge Status

In various embodiments, it would be desirable for the earring devices to be capable of “fast charging” to allow from 85% up to a full battery charge to be obtained within 2 hours of charging (from complete charge depletion or “low battery” status). More preferably, the earring devices are desirably capable of “fast charging” to allow from 85% up to a full battery charge to be obtained within twenty (20) to thirty (30) minutes or ten (10) minutes of charging (from complete charge depletion).

In one exemplary embodiment, a case can incorporate one or more of the following features:

Safely recharge two (2) earring devices simultaneously or one earring individually (if the other earring is being worn, for example)—with capacity to charge individual earrings at differing rates (e.g., fast charge for one earring and trickle or maintenance charge for the other earring):

Contain an internal rechargeable backup battery within the case for powering charger (and charging earrings) when case is not attached to an external power source:

Include a USB Type C power port and charge controller circuit for recharging internal backup battery and/or direct charge to earrings:

Include various internal and/or externally visible LED Status Indicators (e.g., Independent Earring Charge Status, Charge Power Ready, Internal Back-up Battery Charge Status, linked device status, etc.) or alerts including vibrations, noise and/or light notifications.

Incorporate a hinged cover lid (which may optionally include a magnetically attached, hinged cover); and/or

Include space such as depressions or wells for earring closures/backings.

### Wireless Communications And Device Linking

In various embodiments, the disclosed devices utilize wireless communications modalities such as Bluetooth to transmit data (e.g., real time and/or stored data-including two-way data transmission) between the earring device(s) and a computing service such as a wearer's smart device, smart phone and/or other computing device/antenna system with minimal power usage. Such wireless communications can include Bluetooth transmission/reception capacity up to 50 ft in separation (e.g., Bluetooth Low Energy or BLE), but additional wireless communications modalities are contemplated herein, including Wi-Fi and/or cellular connections. In addition, the employment of “airplane mode” and/or limited power consumption modes are contemplated herein.

One significant feature of the present invention is the ability of the earring devices to link to one or more devices such as a mobile phone carried by the wearer and/or to a computing system accessible to the wearer. Desirably, the earring devices can incorporate low-power wireless communications devices such as a Bluetooth BLE 5 communication component which can transmit/pair/link with a mobile or stationary computing devices with Mobile Device Application program and provide sensed data from the earring device to the mobile/stationary computing device. In a preferred embodiment, the mobile/stationary computing device can include a program or computing application (e.g., APP) which can receive, store and analyze data from the earring device(s) and provide the wearer and/or other individuals with a wide variety of health metrics and/or conditions regarding the wearer of the earring(s). In various embodiments, the earing devices may also be capable of receiving data via the Bluetooth or other communications systems, which may alter the functionality and/or performance of the earrings to some desire extent.

In some embodiments, a data logging and/or storage capacity may be included in the earring device(s), especially where real-time data transmission to a computing device is not possible (i.e., the user's smart device is not in BLE range or in airplane mode).

In various embodiments, the device will desirably be EMF-safe, and/or have a capability to enter an EMF-safe mode (which may be automated or may be user controlled using the associated APP). When in such a mode, the device will desirably continue collecting and/or logging data as directed by the user for eventual transmission/downloading to the Application at some future point.

### Applications Programming

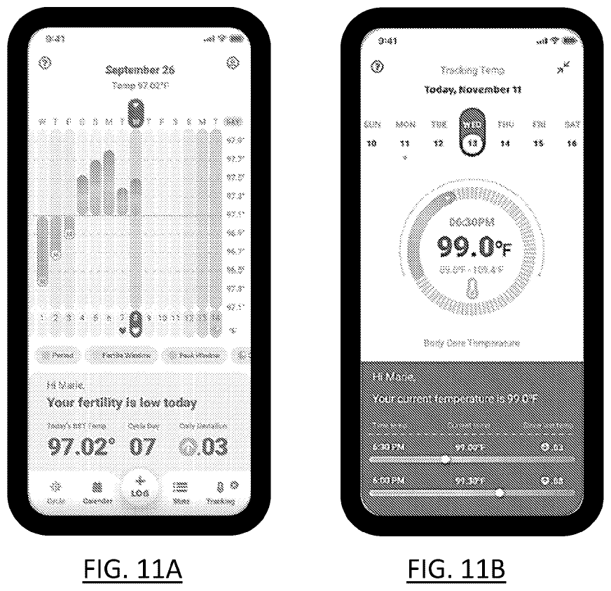

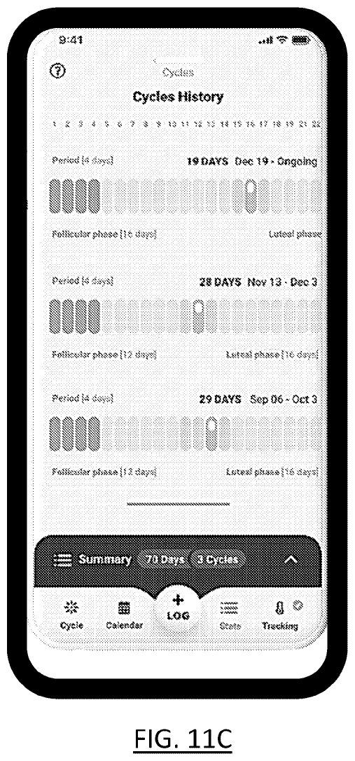

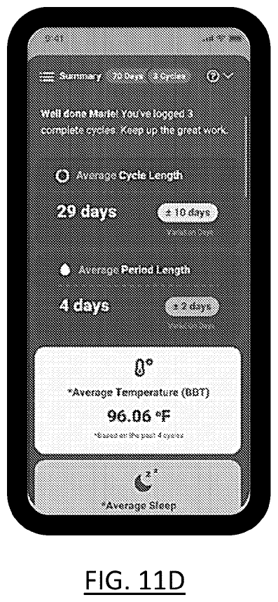

FIGS. 11A through 11D depict various graphical user interface displays (GUI's) for exemplary embodiments of an application program or APP loaded on a mobile phone (e.g., a Samsung S22+ mobile phone, commercially available from Samsung Electronics Corporation of America. In these embodiments, data received from a wearer's sensing device has been transmitted to a mobile phone of the wearer, which includes an application program (APP) which received and analyzes the data and provides processed data summaries to the wearer. Desirably, much of the process of collecting the underlying data can occur without requiring active intervention of the wearer-but is rather automated to some significant degree. In addition to presenting processed and/or raw data, the various GUI's desirably provide various “interpretations” of the data, such as a clinical health or wellness condition such as fertility of the wearer (FIG. 11A), core body temperature (FIG. 11B) and/or menstrual cycle history (FIGS. 11C and 11D).

For example, the disclosed system components can allow user to (1) monitor key metrics about their menstrual cycle alongside stress, activity, and sleep, (2) receive personalized insights and actionable feedback based on their unique metrics, (3) gain insight into their fertility, menstrual cycles, and overall wellness, (4) track their menstrual cycle phases so the user can align their lifestyle accordingly, (5) find the user's fertile window each month to take control of their reproductive health, (6) track periods of high stress for the user to build resilience in the challenges of everyday life, (7) discover activity data and trends to maximize the wearer's performance and results, and/or (8) identify the habits and routines that the wearer needs to find their optimal health, wellness and/or sleep.

The programming application resident on the computing device desirably receives and records the biodata (i.e. CCT temperature, ambient temperature, heart rate, HRV, pulse, breathing rate, blood oxygenation, 3-axis accelerometer) from the earring via Bluetooth technology. The app coding graphs the temperature measurement over time for each night sleep and assessed for the presence of visually identifiable nadir in >50% of waveforms, a measure of this technique's relationship to known physiology. The date of ovulation will be calculated using the inpiercing measurements and controlled against a wearer's artificial intelligence (AI). The cosinor method is used for the coding and mathematical calculations. The app may be resident on a cell phone or similar device having the capability to receive and/or monitor the bluetooth transmission from the earring(s). In various embodiments, a single earring may be utilized, although two earrings may be preferred to detect and/or address temperature variations across the ears (i.e., such as when the wearer may sleep on her side, if an earring falls out, if the wearer's side is turned towards a fan, etc.)

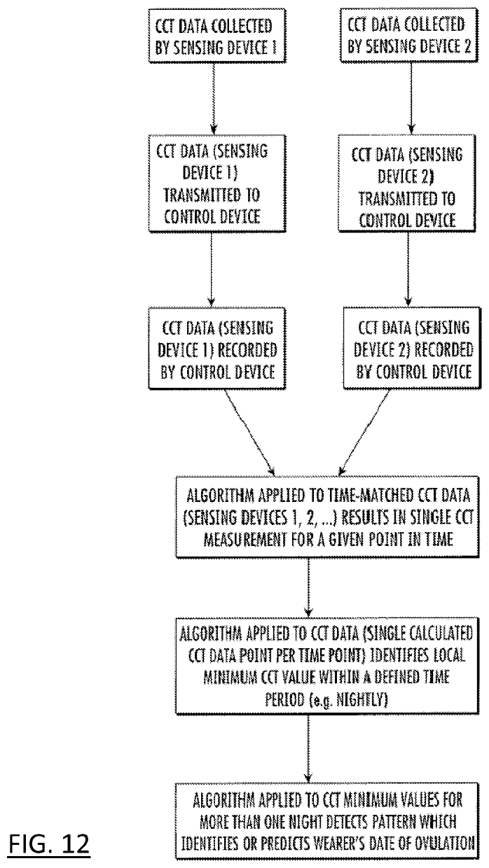

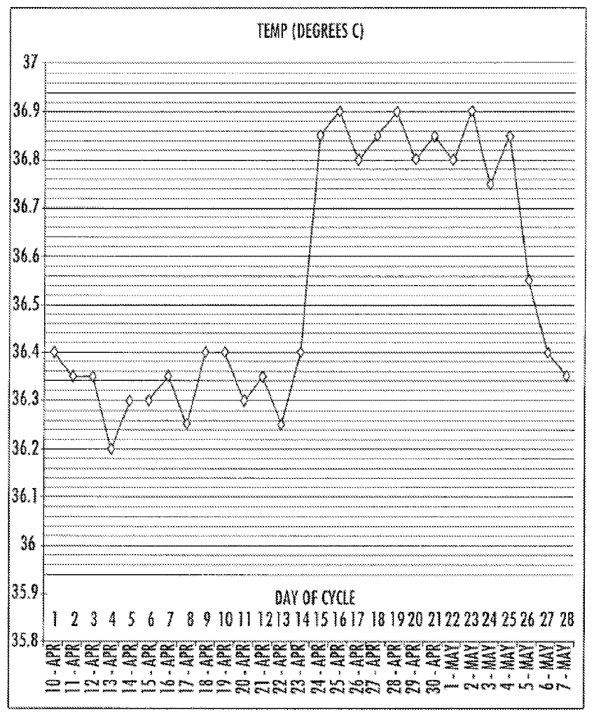

FIG. 12 illustrates one non-limiting flow chart of data processing applied to CCT measurements or values by the Application. When temperature is sensed, it is transmitted, and then recorded wirelessly by a receiver. The receiver records CCT in a format that desirably retains the CCT value, which sensing device it came from (used to distinguish between different devices when using more than one device at once), and the time. Two or more wearable sensors and/or temperature sensors can be utilized with separate created cavities within one body. When more than one wearable sensor and/or temperature sensor is used at once, the receiver can obtain all temperature readings. These temperatures can be compared and an algorithm can be applied to arrive at one temperature value for that time. For example, but not limited to, one sensing device is used in the left ear with another sensing device used in the right car. In a non-limiting example, when the receiver obtains both temperature readings, the lowest temperature value is chosen for use in additional calculations.

In one exemplary embodiment, the recorded temperature values can be plotted against time and an algorithm applied to find local and global minima and maxima. Further, global minima for a given time period can be plotted against sequential time periods. For example, but not limited to, using the minimum CCT for a given night, which is then implemented across many nights. With applying the algorithm to plot CCT for many nights, quantified patterns are determined and ovulation trends are revealed. One can then draw conclusions derived from ovulation patterns to determine or predict outcomes for physiological consequence and/or state.

The main algorithm is used to determine local and global minima and maxima: although, various optional algorithms can be employed. A non-limiting example of this is seen where an algorithm is used to calculate IBT or BBT by measuring CCT for assistance in determination of fertility.

FIG. 13 graphically illustrates the use of temperature measurements in relation to time for determination of fertility. It is known in the art that determining when ovulation occurs can be verified by means of tracking the elevation in body temperature during a woman's fertility cycle, where this temperature rise comes abruptly at the time of ovulation. This temperature rise is caused due to the secretion of progesterone during the latter half of the cycle, which increases the body temperature about one-half degree Fahrenheit. During the first half of the menstrual cycle, the temperature fluctuates around 97.2 to 98.0 degree Fahrenheit, and then in a space of 1-2 days, the temperature undergoes a rather steep rise of about one-half or more degrees Fahrenheit, to around 98.2 to 99.0 degree Fahrenheit. The temperature remains at this higher level until the next menstrual bleeding. With this information, one can extract that, on average, ovulation occurs 1-2 days before the steep rise in temperature. This small increase in temperature explains the need for a temperature sensor that accurately detects small, 0.1 to 0.01 degree Fahrenheit, changes.

### Artificial Intelligence (AI) Processing

In various embodiments, the measured biodata from the earring can be processed using an artificial intelligence module, which will desirably mine the temperature data (and other data received from the device as well as other information obtained by the AI regarding the wearer's activities or daily living data). The AI module will desirably correlate and compare prior CCT and other data in an attempt to identify relationships and trends between data sets, such as elevated body temperature during periods of exercise (which might induce an artificially high body temperature for a period of time which mimics an ovulation temperature increase), or similar indicators. Desirably, the AI module will have access to wearer data outside of the data sent to the module from the earring. In addition, over a period of weeks or months of data collection, the AI may be able to identify unique indicators for the wearer's health that would not be noticeable to a physician under normal circumstances, which may help to reduce false positives/negatives as well as potentially increase the sensitivity and/or accuracy of the data presented by the application and/or early detection of on-set conditions or diagnosis.

Desirably, the disclosed devices can be made to be worn in a manner as simple as wearing an earring, with data transferred to the Application resident on a cell phone that can be carried by the wearer and/or software platform for remote patient monitoring, healthcare and clinical monitoring and notifications. With its small, compact design, this wearable sensor is intended for continuous wear during normal daily or nightly activities. The size, comfort, wearer-friendliness, and non-manual operations allow the wearer to not interrupt daily activities or awaken while the device is reading the wearer's useful temperature of CCT or other metrics. Further, the CCT information and/or other metrics can be wirelessly sent to an external processor or memory device for tracking temperature and/or other metrics.

In addition to the collection, analysis, storage and presentation of user data, the application will desirably include additional features to ensure the ability to update and/or improve the performance of the earring devices, such as the capacity for automatic firmware updates via the data application. Similarly, it is highly desirable for the application to have the ability to push software updates and/or incorporate improvement into the devices, such as an ability to add HRV, oxygen, accelerometer and/or other health measurements to an existing earring design, either via software updates or additional components/upgrades.

Smart Closure/Backing Embodiment

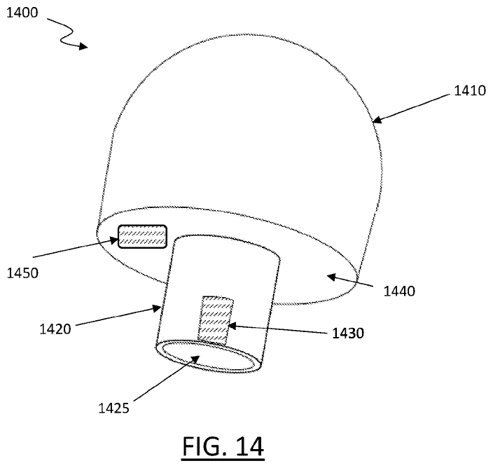

FIG. 14 depicts an alternative embodiment of the disclosed wearable device which can be used as a closure, backing or similar attachment device to a multiplicity of earrings, including earring that the wearer may already possess prior to acquisition and/or usage of the disclosed devices. In this embodiment, the wearable sensor device 1400 comprises a central body 1410 with associated electronic components (and an optional cover) similar to those previously described, but this device includes a post or sleeve 1420 having an opening 1425 formed therein which is desirably capable of engaging with and securing to a standard or regular earring post, which typically ranges from 21 to 18 gauge in diameter (or approximately 0.71 mm to 1.0 mm or 0.028 inches to 0.040 inches). If desired, the sleeve can comprise a rigid structure or relatively flexible/expandable material to allow a plurality of earring post gauges to be accommodated and/or secured therein. Desirably, the device 1400 will include at least one sensor, biochemical sensor, electrical sensor, electrochemical sensor, electromechanical sensor, electrodes, optical, and/or temperature sensor or thermistor 1430 on an exposed portion of the sleeve 1420. Optionally, in an embodiment, the device 1400 will further include at least one sensor, biochemical sensor, electrical sensor, electrochemical sensor, electromechanical sensor, electrodes, and/or optical sensor 1450 on an exposed portion of the base 1440. Sensors may include but are not limited to accelerometer. Optical sensors, biochemical sensors, bioelectrical sensors, electrochemical sensors, pressure sensor, CGM sensor, Gyroscope, GPS and other wearable sensors, LED sensors for heart rate, HRV, breathing rate, blood oxygenation, pulse, Accelerometer for movement, activity, calories, distance traveled, steps, LED sensor and Pressure sensor for blood pressure, CGM sensor for glucose monitoring, Gyroscope for angular velocity measurements, Global positioning system (GPS), Magnetometer for magnetic measurements, electrochemical sensors for hormone and other biosensing and Speaker and/or Microphone for music, phone calls.

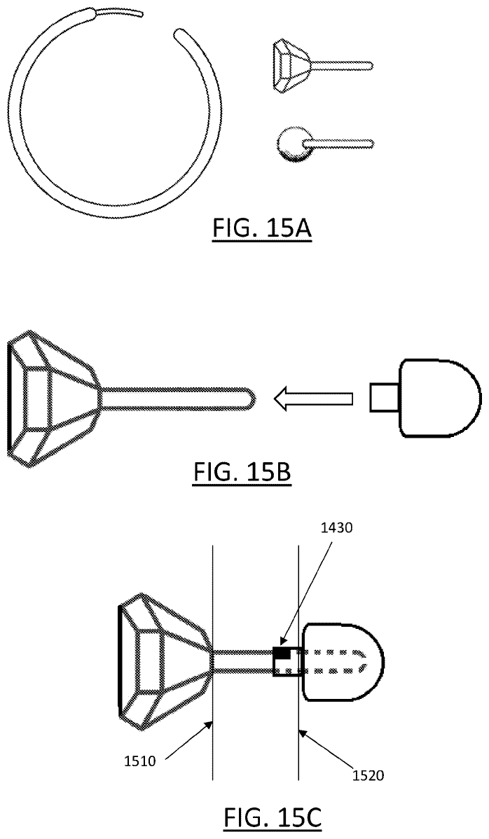

As best seen in FIGS. 15A through 15C, the disclosed backing sensor device 1400 may be compatible or can be utilized with a variety of earring shapes and/or types (FIG. 15A). The backing sensor device can be placed over the earring post in a sleeved or jacketed manner or surrounding the post of the earring (see FIG. 15B), and then the sleeve will desirably travel along the post with the sleeve and sensor desirably entering the created cavity (depicted as a first skin wall 1510 and a second skin wall 1520 of the ear) from the backside of the ear (with the sleeve and sensor desirably being thin enough to slip between the post and the cavity wall during use-see FIG. 15C), thereby providing an accurate measurement of the created cavity temperature and other biosensor measurements as described previously. Similarly, the base of the central body base 1440 may include additional sensing components, lights, etc. as previously described (as well as open and/or transparent sections to accommodate such components) to allow measurement and assessment of the various wearer characteristics as described herein.

It should be understood that this alternative embodiment may include any of the previously described components and/or capabilities, as well as associated charging case components and applications software for linking to computing devices. In this manner, a wearer may utilize the system components with a variety of earring designs, which may be switched daily or even more frequently without disturbing the data collection and analysis as described herein.

If desired, the closure device may be configured to function with earring posts of differing types, including post, push back stud, screw post, hinged hoop, latch back, lever, spring, huggie, French clip and/or other clasp types known to those in the art.

### Equivalents

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A sensing device for continuously monitoring a physiological condition of a wearer having an artificially-created cavity in living body tissue, the artificially-created cavity being bounded by a first hole in body tissue and a second hole in body tissue opposite the first hole, the sensing device comprising:
a central body having a first sensor component positioned outside of the artificially-created cavity at a location adjacent to a skin surface of the wearer, the first sensor providing a first signal which is a measurement of a first physiological condition of the wearer continuously or periodically using specified time intervals, and
a body piercing device post, the body piercing device post being configured for insertion and to be worn in the artificially-created cavity, said body piercing device post being dimensioned to be inserted through at least the first hole of the body tissue and into the artificially-created body tissue cavity, the body piercing device post containing a second sensor component positioned within the artificially-created body tissue cavity and providing a second signal which is a measurement of a second physiological condition of the wearer continuously or periodically using specified time intervals;
a transmitter configured to wirelessly transmit said first and second measurements continuously or periodically to a remote receiver for recording, the transmitter being located within the central body such that in use it is located outside of the artificially-created cavity; and
the sensing device further comprising at least one power supply that powers said first and second sensors and said transmitter,

2. The sensing device of claim 1, wherein the first sensor measures blood oxygenation of the wearer and the second sensor measures temperature of the wearer,

3. The sensing device of claim 1, wherein the first sensor measures blood pressure of the wearer and the second sensor measures temperature of the wearer,

4. The sensing device of claim 1, wherein the first sensor measures blood oxygenation of the wearer and the second sensor measures glucose levels of the wearer,

5. The sensing device of claim 1, wherein the sensor is removeable by the wearer,

6. A sensing device for continuously monitoring a physiological condition of a wearer having an artificially-created cavity in living body tissue, the artificially-created cavity being bounded by a first hole in body tissue and a second hole in body tissue opposite the first hole, the sensing device comprising:
a central body having a first sensor component positioned outside of the artificially-created cavity at a location adjacent to a skin surface of the wearer, the first sensor providing a first signal which is a measurement of a first physiological condition of the wearer continuously or periodically using specified time intervals, and
a sleeved tube for receiving a body piercing device post extending through the artificially-created cavity, the sleeved tube including an extending portion which substantially surrounds the post and passes into the artificially-created cavity, the extending portion containing a second sensor component positioned within the artificially-created body tissue cavity and providing a second signal which is a measurement of a second physiological condition of the wearer continuously or periodically using specified time intervals;
a transmitter configured to wirelessly transmit said first and second measurements continuously or periodically to a remote receiver for recording, the transmitter being located within the central body such that in use it is located outside of the artificially-created cavity; and
the sensing device further comprising at least one power supply that powers said first and second sensors and said transmitter.

7., The sensing device of claim 6, wherein the first sensor measures blood oxygenation of the wearer and the second sensor measures temperature of the wearer,

8. The sensing device of claim 6, wherein the first sensor measures blood pressure of the wearer and the second sensor measures temperature of the wearer,

9, The sensing device of claim 6, wherein the first sensor measures blood oxygenation of the wearer and the second sensor measures glucose levels of the wearer.

10. The sensing device of claim 6, wherein the sensor is removeable by the wearer.
